(19) United States
(12) Plant Patent Application Publication
(10) Pub. No.: US 2015/0040274 P1
(43) Pub. Date: Feb. 5, 2015

(54) Prunus tree rootstock named 'Gi31817'

(50) ;

(71) Applicant: Consortium Deutsche Baumschulen GmbH, Ellerbek (DE)

(72) Inventor: Sabine Franken-Bembenek, Giessen (DE)

(73) Assignee: Consortium Deutsche Baumschulen GmbH, Ellerbek (DE)

(21) Appl. No.: 13/987,521

(22) Filed: Aug. 1, 2013

(51) Int. Cl. A01H 5/00

(52) U.S. Cl. PLT/180

(57) ABSTRACT

A new and distinct Prunus hybrid plant used as a rootstock for cherries that induces precocity and flat branching of the grafted plant.

### BOTANICAL CLASSIFICATION

Prunus hybrid

### VARIETAL DENOMINATION

‘Gi31817’

### BACKGROUND OF THE INVENTION

The present invention comprises a new and distinct cultivar of Prunus hybrid used as a rootstock known by the varietal name ‘Gi31817’. The new variety was discovered in Giessen, Germany in 1970. The new variety is the result of a planned breeding program between a Prunus canescens (female parent) and Prunus avium Vogelkirsche, mazzard (male parent). The purpose of the breeding program was to produce a series of size-controlling, productive, and precocious rootstocks for sweet cherries. The new variety has been trial and field tested and has been found to retain its distinctive characteristics and remain true to type through successive propagations.

The new variety is similar to Prunus rootstock ‘GI 148/2’ (U.S. Plant Pat. No. 9,622) in its rootstock induction of precosity, rootstock induction of flat branching, and the absence of root suckers. The leaves of the new variety have a different appearance than those of ‘GI 148/2’. Further, the vigor of a grafted tree on the new variety is much greater than on ‘GI 148/2’.

### DESCRIPTION OF THE DRAWINGS

The accompanying photographic drawings illustrate the new cultivar, with the color being as nearly true as is possible with color illustrations of this type:

FIG. 1 is a close-up picture of the leaves and branches of the new variety; and

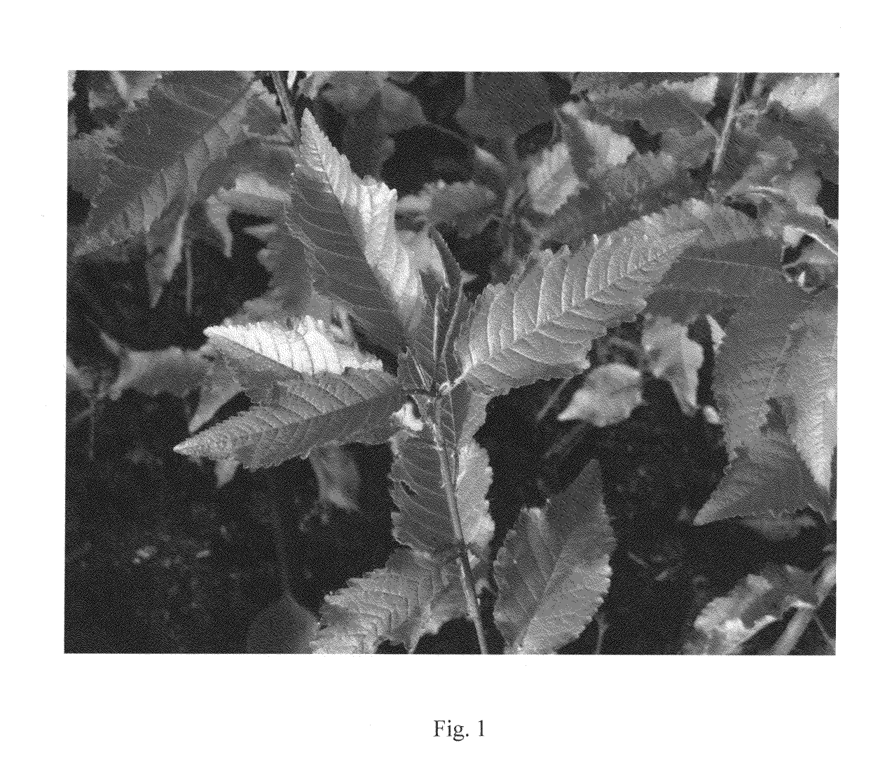

FIG. 2 is a picture of a numerous plants of the new variety.

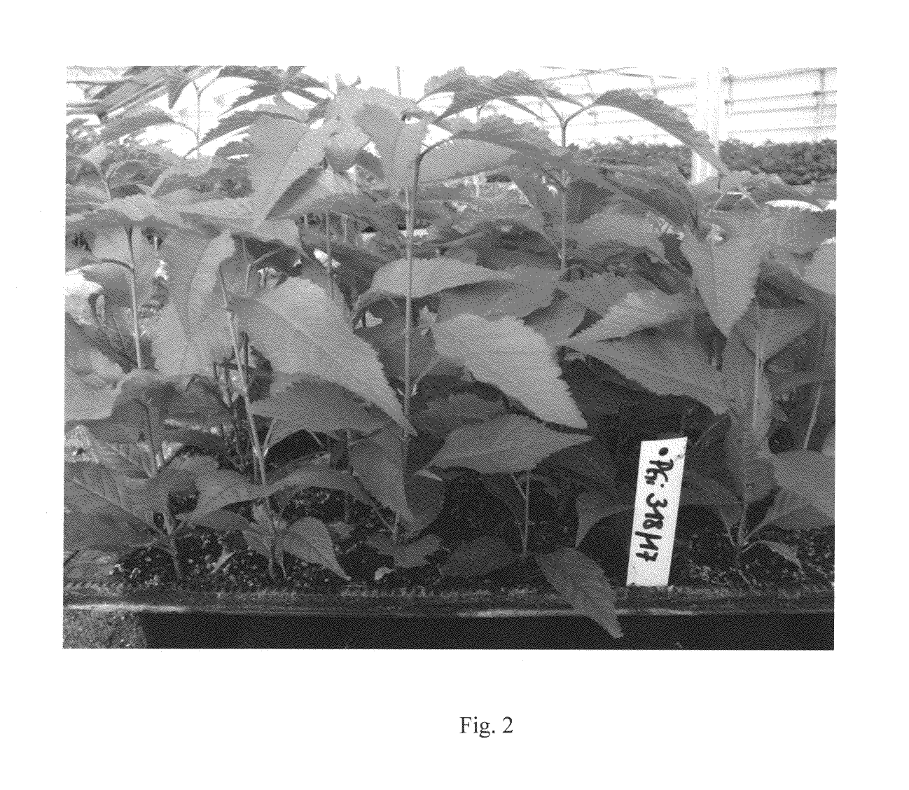

### DESCRIPTION OF THE PLANT

The following detailed description sets forth the characteristics of the new cultivar. The data which defines these characteristics were collected by asexual reproduction carried by green cuttings under mist and in vitro conditions. The first asexual reproduction occurred in approximately 1975 in Giessen, Germany. The color readings were taken in natural daylight. Color references are primarily to the R.H.S. Colour Chart of The Royal Horticultural Society of London.
Plant:
   Use.—As a rootstock for sweet, sour, and ornamental cherries.
   Growth type.—Upright.
   Pathogen resistance.—Fungal disease: Tolerant to Monilia. Viruses: Tolerant to ilarviruses PDV and PNRSV.
   Branch color.—175B.
   Leaves.—Length: Medium. Width: Medium. Form: Elliptic. Apex: Acute. Base: Obtuse. Margin: Serrate. Pubescence: Weak presence on lower surface. Color: Upper surface: 137A. Lower surface: 137C. Petiole length: 1.0 cm.
   Flowers.—Pedicel length: 1.5 cm. Bud color: 56D. Bloom timing: Similar to most sweet cherry cultivars, like ‘Hedelfinger’. Petals: Number: Five. Length: 0.8 cm. Shape: Round. Color (fully opened): White on both surfaces. Sepals: Length: 0.4 cm. Color: 144A.
   Tree winter hardiness.—Good.
   Performance as a grafted rootstock.—Root sprouts: No suckers observed. Anchorage: Good. Compatibility: Good. Vigor: Reduces size of tree compared to Prunus avium rootstock, much more vigorous than ‘GI 148/2’, more vigorous than ‘GI 148/1’.

I claim:

1. A new and distinct variety of Prunus hybrid plant used as rootstock as shown and described herein.
